(19) United States
(12) Patent Application Publication
(10) Pub. No.: US 2001/0044393 A1
(43) Pub. Date: Nov. 22, 2001

(54) Rinse-off antimicrobial liquid cleansing composition

(76) Inventors: Robert Frederick Peterson JR., Abington, PA (US); Larry Kent Hall, Nazareth, PA (US)

Correspondence Address: DARBY & DARBY, 805 THIRD AVENUE, 27TH FLR., NEW YORK, NY, 10022, US

(21) Appl. No.: 09/810,692

(22) Filed: Feb. 12, 2001

Related U.S. Application Data
(60) Provisional application No. 60/183,769, filed on Feb. 18, 2000.

(51) Int. Cl. A61K 7/50

(52) U.S. Cl. 510/130; 510/382; 510/384; 510/504

(57) ABSTRACT

An aqueous antimicrobial liquid cleaning formulation comprising about 0.2 to 4 parts of a cationic quaternary ammonium compound; about 2 to 10 parts of one or more nonionic surfactants; and about 1 to 10 parts of a polyfunctional alcohol wherein the weight ratio of nonionic surfactant and polyol to cationic surfactant is at least about 4:1 to about 28:1.

### FIELD OF THE INVENTION

This invention relates to a novel rinse-off antimicrobial liquid cleansing composition that is non-irritating to the skin, comprising a cationic quaternary ammonium surfactant, a nonionic surfactant and a polyalcohol, wherein the ratio of nonionic surfactant and polyalcohol to cationic quaternary ammonium surfactant is at least 4:1 to about 28:1.

### BACKGROUND OF THE INVENTION

Antimicrobial cleansing products have been marketed in a variety of forms including solid and liquid antimicrobial hand soaps, hard surface cleaners, and surgical disinfectants. These products are typically formulated with antimicrobial agents and surfactants to remove bacteria during washing and to provide a residual effectiveness against certain bacteria. In formulating these compositions, care must be taken to avoid mixing components that inactivate the antimicrobial agent. For example, certain types of surfactants, included in these formulations as cleansing agents, inhibit the bioactivity of antimicrobial agents.

Many antimicrobial cleansing compositions contain a phenolic antimicrobial agent such as triclosan, p-chloro-m-xylenol, or phenoxyethanol. Phenol-derived antimicrobial agents are described in U.S. Pat. No. 5,968,539 to Beerse et. al, U.S. Pat. No. 5,942,478 to Lopes, U.S. Pat. No. 5,837,274 to Shick and Wheeler, and in U.S. Pat. No. 4,939,284 to Degenhardt. To avoid the inactivation of the phenolic antimicrobial agent by cleansing agents, these formulations include a mixture of anionic, nonionic and amphoteric surfactants with the phenolic antimicrobial agent. Beerse et. al. discloses that “non-cationic actives are required in order to avoid interaction with the anionic surfactants of the invention.”

Another problem arises in that many antimicrobial agents are added to cleaning compositions at concentrations that, although effective in killing microbes, cause irritation to the skin. This can be problematic especially when the compositions are used frequently, where the control of transmission of infection or pathogens is desired for example in medical or food service environments.

In order to avoid skin irritation by harsh phenol-derived antimicrobial agents, surfactants may be added to provide protection to the skin against these harsh chemicals. However, as mentioned above, surfactants can inactivate the antimicrobial agent. Shick and Wheeler disclose the efficacy of a phenol derivative antimicrobial agent in combination with a surfactant system comprising predominantly nonionic surfactants. However, in order to maintain effective antimicrobial activity and to avoid skin irritation, this system further included a polymeric deposition aid comprised of liquid hydroxyl-terminated urethane polymers in polyethylene.

Quaternary ammonium compounds are also used as antimicrobial agents. The use of quaternary ammonium antimicrobial agents, such as benzethonium chloride and benzalkonium chloride, in antimicrobial cleansing products is limited because of the lack of compatibility of these compounds with other ingredients commonly used in cleansing products. The effect of anionic surfactants, for example, may be so great as to eliminate completely most measurable antimicrobial activity. Many nonionic surfactants also reduce or eliminate the activity of quaternary ammonium surfactants; in fact, the general testing procedure for biocidal efficacy makes use of the deactivating effects of nonionic sorbate esters. Due to the interaction between nonionic surfactants and quaternary ammonium compounds, a ratio of lower than 4:1 of nonionic to cationic surfactant is suggested to avoid deactivation of quaternary ammonium compounds (Davis, Critical Reports on Applied Chemistry, Vol.30, (ed. M. R. Porter), Elsevier, 1990, ISBN 1-85166-475-0).

An effective antimicrobial cleansing composition, that gently cleanses the skin, causes little or no skin irritation, and preferably conditions the skin, has not heretofore been easily obtained.

### SUMMARY OF THE INVENTION

The present invention provides an aqueous rinse-off antimicrobial cleansing solution comprising from about 3 to 25 wt % of non-aqueous constituents. The non-aqueous constituents comprise from about 0.2 to 4 wt % of a cationic surfactant; from about 2 to 10 wt % of a nonionic surfactant; and from about 1 to 10 wt % of a polyalcohol (hereinafter referred to as a “polyol”). The percentages refer to the amount of active constituents in the aqueous solution.

The present invention further provides an aqueous antimicrobial composition having non-aqueous constituents which comprise from about 0.2 to 4 parts of cationic surfactant; about 2 to 10 parts of a nonionic surfactant; and about 1 to 10 parts of a polyol.

The ratio of the combination of nonionic surfactant and polyol to cationic surfactant in the non-aqueous composition is at least 4:1 to about 28:1. The non-cationic components of the non-aqueous constituent, comprising nonionic surfactant and polyol, are hereinafter referred to as “non-cationic surface active agents”. It has been found that this ratio of non-cationic surface active agents to cationic surfactant present in the non-aqueous constituent unexpectedly maintains a favorable balance of biocidal efficacy and mildness to skin not found in currently available antimicrobial cleansing compositions.

In the present invention, the cationic surfactant is a quaternary ammonium compound, preferably benzalkonium chloride or benzethonium chloride. The nonionic surfactant is preferably an alkyl polyglucoside, an ethoxylate of a linear alcohol having twelve or less carbon atoms or an amine oxide having an alkyl group having from 8 to 14 carbon atoms. The polyol is preferably glycerol, polyglycerol, sorbitol and hydrogenated starch hydrolysate; preferably the polyfunctional alcohol is glycerol or decaglycerol.

### DETAILED DESCRIPTION OF THE INVENTION

The percentages of the components of the non-aqueous constituents of the present invention based on the total aqueous composition are provided in Table 1 below. All percentages and ratios used herein are by weight, unless otherwise indicated.

TABLE 1

| | | Broad range | Preferred range |
|---|---|---|---|
| Constituent | Function | wt % | wt % |
| Cationic Surfactant | Antimicrobial | 0.2-4 | 0.5-2 |
| Nonionic Surfactant | Cleanser | 2-10 | 5-6 |
| Polyol | Skin Conditioner | 1-10 | 5-7 |

The ratio of the amount of non-cationic surface active agents to the amount of cationic surfactant present is at least 4:1 to about 28:1, preferably 4:1 to about 24:1, and most preferably is about 20:1.

The present invention comprises a cationic surfactant as the antimicrobial active. In the present invention, the preferred cationic surfactant is a quarternary ammonium compound. Suitable quarternary ammonium compounds include benzethonium chloride, benzalkonium chloride, dialkyl dimethyl ammonium compounds; alkylbenzyl ammonium compounds; and cetrimide (alkyltrimethyl ammonium bromide).

The quaternary ammonium compounds have the formula

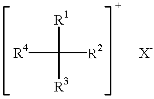

wherein R¹ and R² are independent C₁-C₆ alkyl or hydroxyalkyl, R³ and R⁴ are independently linear or branched C₈-C₃₀ alkyl, C₆-C₂₀ aryl-substituted alkyl, and X is an anion. Preferably, R¹ and R² are independently C₁-C₃ alkyl or hydroxyalkyl and more preferably methyl. R³ is preferably a linear or branched C₈-C₁₈ alkyl or benzyl. R⁴ is preferably a linear or branched C₈-C₁₈ alkyl. X is preferably a halogen, carbonate, phthalate or propionate.

Examples of the quaternary ammonium compounds include [2-[2-(4-diisobutylphenoxy)ethoxy]ethyl] dimethyl benzyl ammonium chloride (also known as benzethonium chloride or Hyamine® 1622); a dialkyl (C₈ to C₂₂) dimethylammonium chloride; a dialkyl (C₈ to C₂₂) methyl poly(oxyethyl) ammonium chloride; an alkyl (C₈ to C₂₂) benzyldimethyl ammonium chloride; an alkyl (C₈ to C₂₂) trimethylammonium chloride; a dialkyl (C₈ to C₂₂) dihydroxyethyl ammonium chloride; or an alkyl (C₈ to C₂₂) methyl dihydroxyethyl ammonium chloride; N,N-didecyldimethylammonium chloride (available as Bardac® 22); N,N-decylisononyl dimethyl ammonium chloride (available as Bardac(® 21); N,N-decyloctyl dimethyl ammonium chloride (available as Bardac® 20); N,N-dioctyldimethyl ammonium chloride (available as Bardac® LF); hexadecyltrimethyl ammonium chloride (available as Barquat® CT-29); octadecyldimethylbenzyl ammonium chloride (available as Carsoquat® SDQ-25); N,N-didecyl-N-methyl-poly(oxyethyl) ammonium propionate (available as Bardap® 26); or alkyl (C₁₀-C₁₈) dimethyl benzyl ammonium chloride (available as Barquat® CB-50).

The present invention further comprises a nonionic surfactant as a cleansing agent. Nonionic surfactants suitable for the present invention include alkyl polyglucosides, alkyl phenol ethoxylates, alcohol ethoxylates, amine oxides and polyglycerol esters. Preferably, the nonionic surfactant is an alkyl polyglucoside, an ethoxylate of a low molecular weight linear alcohol having 12 or less carbons, or an amine oxide having an alkyl group of 14 or less carbons, preferably 8-14 carbons. Useful amine oxides include octyldimethylamineoxide, decyl dimethyl amine oxide, and dodecyl dimethyl amine oxide. Nonlimiting examples of nonionic surfactants useful in the compositions of this invention are described in McCutcheon's Detergents and Emulsifiers, North American Edition published yearly by The Manufacturing Confectioner Publishing Company.

The antimicrobial cleansing composition of the invention also comprises a polyol as a skin conditioner to produce a composition that is non-irritating to the skin. Suitable polyols that can be used in the composition of the invention include glycerol; oligomers of glycerol such as triglycerol, pentaglycerol and decaglycerol (polyglycerols); sorbitol; and hydrogenated starch hydrolysate.

The non-aqueous component of the present invention may optionally comprise a wide variety of other non-aqueous ingredients commonly used in the skin care industry and are well known to one of ordinary skill in the art. Nonlimiting examples of such ingredients include thickeners, chelants, preservatives, carriers, pH regulators, dyes and fragrances such as those commonly used in skin cleansing solutions.

The cleansing composition of the invention is effectively microbiocidal without the adjustment of the pH. However, the pH of the solution can be advantageously adjusted with common pH regulating agents such as alkaline agents, e.g., alkaline metal hydroxides, and by utilizing appropriate buffering agents such as EDTA, to a pH of from 6 to 8.5.

Aqueous antimicrobial rinse off compositions according to the present invention may be prepared by simply mixing all the components of the non-aqueous constituent of the invention with the desired aqueous diluent, such as water, until a uniform mixture is formed. Nonaqueous diluents may also be included such as methanol, ethanol, isopropanol and glycol ethers.

Abbreviations and/or trade names of compositions or compounds used herein and the corresponding compositions or compounds to which they refer are listed in Table 2.

TABLE 2

| Abbreviation | Trade name | Composition |
|---|---|---|
| | | Cationic surfactant |
| BACl | Barquat | Benzalkonium chloride (50% active solution) |
| BzCl | Hyamine 1622 | Benzethonium chloride (50% active solution) |
| | | Nonionic surfactant |
| Bar 12 | Barlox 12 | N-dodecyl dimethyl amine oxide (30% solution) |
| — | Neodol 1-5 | PEO 5 C11 linear alcohol |
| — | Neodol 1-7 | PEO 7 C11 linear alcohol |
| N 1-9 | Neodol 1-9 | PEO 9 C11 linear alcohol |
| — | Neodol 91-2.5 | PEO 2.5 (C9-C11) linear alcohol |
| — | Neodol 91-6 | PEO 6 (C9-C11) linear alcohol |
| — | Neodol 25-7 | PEO 7 (C12-C15) linear alcohol |
| N25-12 | Neodol 25-12 | PEO 12 (C12-C15) linear alcohol |
| P818 | Plantaren 818 | Alkyl polyglucoside |
| 10-1-O | Polyaldo 10-1-O | Decaglycerol monooleate |
| 3-0.8-L | Polyaldo 3-0.8-L | Triglycerol (0.8) laurate |
| — | Tergitol 15-S-7 | PEO 7 C15 secondary alcohol |
| — | Tergitol 15-S-12 | PEO 12 C15 secondary alcohol |
| — | Tergitol 15-S-20 | PEO 20 C15 secondary alcohol |
| TDA-6 | | PEO 6 trimethyldecyl alcohol |
| | | Polyol |
| Deca-g | Decaglycerol | Decaglycerol |
| Gly | Glycerol | Glycerol |
| Hystar | Hystar CG | Sorbitol (40% solution) |
| | | Other |
| Methocel | Methocel J12MS | Methylcellulose ether, thickener |
| | Methocel 40-100 | |
| | Methocel 40-101 | |
| | Methocel 40-202 | |

The cleansing composition of the present invention advantageously provides a rinse-off liquid cleansing composition effective for cleansing surfaces, especially the skin, of dirt, bacteria and oil. It successfully maintains the desired antimicrobial activity of the quaternary ammonium compound against a range of bacterial species, including Staphylococcus aureus, Escherichia coli and Staphylococcus marcescens, even in the presence of the non-cationic surface active agents, and is non-irritating to the skin, as measured, for example, by patch testing. The composition provides a “good feel quality” to the liquid cleanser composition, as rated by objective panelists. It may be advantageous to combine two or more non-cationic surface active agents and adjust the amounts and proportions of the nonionic surfactants to meet the requirements for specified end use of the composition. It is well within the ability of those of skill in the art, using methods such as those described herein, to determine the efficacy and mildness of the antimicrobial compositions, to select a suitable combination of ingredients, i.e., nonionic surfactants and polyols, which is most compatible with the cationic quaternary ammonium compounds used in the present invention.

The term “rinse-off” refers to a composition that is rinsed or washed off from the treated surface during or after the application of the product. Also contemplated is a product which is not rinsed or washed from the treated surface, such as when the product is formulated as a towelette or wipe. A preferred embodiment of the present invention is a liquid cleansing composition suitable for use on human skin. The cleansing composition of the invention is applied to the skin in a microbiocidal effective amount.

The following Examples illustrate more specifically the invention. It will be understood that while the invention as described therein is a specific embodiment thereof, the description above and the examples are intended to illustrate and not limit the scope of the invention. It will be apparent to those skilled in the art that modifications may be made therein without departing from the spirit and scope of the invention.

### EXAMPLES

### Example 1

This example illustrates a method for preparing four antimicrobial compositions according to the present invention. The Example further provides methods of testing the effectiveness of the compositions in reducing or destroying bacteria on the skin and in culture and in avoiding skin irritation.

A. Preparation of Antimicrobial Compositions

Four compositions having the formulations shown in Table 3 were prepared:

TABLE 3

| | Composition | | | |
|---|---|---|---|---|
| Ingredient | A | B | C | D |
| Cationic Surfactant | | | | |
| Benzethonium chloride (50% solution) | 1.0 | 1.0 | — | — |
| Benzalkonium chloride (50% solution) | — | — | 1.0 | 1.0 |
| Non-cationic | | | | |
| Nonionic Surfactant | | | | |
| Plantaren 818 | 1 | 1 | 1 | 1 |
| Neodol 25-12 | 2 | 2 | 2 | 2 |
| Polyaldo 10-1-O | 1 | 1 | 1 | 1 |
| Barlox 12 (30% solution) | 6.5 | 6.5 | 6.5 | 6.5 |
| Polyol | | | | |
| Glycerol | 5 | — | 5 | — |
| Decaglycerol | — | 5 | — | 5 |
| Methocel J12MS | 1.5 | 1.5 | 1.5 | 1.5 |
| Water | 82 | 82 | 82 | 82 |
| Non-cationic/cationic surface actives ratio | 21.9:1 | 21.9:1 | 21.9:1 | 21.9:1 |

All ingredients except Methocel were mixed at room temperature and stirred until uniform. Methocel was added and the mixture was stirred until thick and uniform.

The antimicrobial activity and skin irritation property of each of compositions A, B, C and D were evaluated and compared to a commercial formulation of Dial Antibacterial Liquid Soap (containing triclosan as the active ingredient and a mixture of anionic and nonionic surfactants), denoted as Composition E, in the tests described below.

B. Modified Cade Test

Each of the compositions A, B, C, D and E was tested on the hands of seven subjects. The subjects did not use any antimicrobial products for five days prior to the start of the test. First, each subject washed their hands with a non-antibacterial soap, e.g., Softsoap. The subjects moistened their hands for 15 seconds in a basin of water. A non-antibacterial soap or test product (1 cc, using sterile syringe) was added; the subjects lathered their hands for 60 seconds; and rinsed for 15 seconds into the basin. The water in the sterile basins was sampled, neutralized immediately in Letheen broth, plated onto Tripticase Soy Agar and incubated at 35° C. for 72 hours. A total aerobic plate count of bacterial colonies was performed at that time. Hand were washed two more times using the non-antibacterial soap; these washes were not saved. On the fourth wash on Day 1 hand were washed with one of the test compositions A, B, C, D, or E described above. On Day 2, the subjects came to the laboratory for the first wash of the day with the test sample. The remaining two washes of the second day, and all three washes on Day 3 were done at home. On Day 4, the subjects returned to the laboratory for a final wash with the test sample. The water in those basins was sampled, neutralized, plated, incubated and counted as described above.

The results were expressed as log reduction (log bacterial colonies after final wash−log bacterial colonies in first wash). The results of the Modified Cade test are shown in Table 4.

TABLE 4
Modified Cade Test

| Composition | Log Reduction |
|---|---|
| A | 1.9 |
| B | 1.1 |
| C | 1.9 |
| D | 1.1 |
| E | 0.3 |

Hand surfaces washed with each of the test formulations A-D had a log or greater reduction in the number of bacterial colonies on the hand surfaces compared to hands washed with the commercial standard Composition E, and thus met the requirements of the invention.

C. Skin Irritation Test

Skin irritation by the formulations was tested on 106 panelists. Each subject's back was washed with a non-bacterial soap and then dried. 0.1 ml of each of the formulations A through E was applied to a cloth bandage which was then secured to the subject's back using adhesive. The bandage was left in place for 48 hours, and then was removed. Immediately after removal, and 24 hours after removal, the degree of skin irritation was rated on a 0 to 4 scale using the following ratings scale:

| Score | Description |
|---|---|
| 0.0 | No evidence of any effect |
| 0.1 | Minimal erythema (reddening) |
| 1.0 | Definite erythema |
| 2.0 | Erythema and edema (swelling) |
| 3.0 | Erythema with vesiculation and edema |
| 4.0 | Intense erythema with bullae (visible pustules) |

The results shown in Table 5 represent the average ratings of each formulation tested in the above manner.

TABLE 5

Skin Irritation Score

| Formulation | 48 hours | 72 hours |
|---|---|---|
| A | 0.04 | 0.06 |
| B | 0.005 | 0.02 |
| C | 0.02 | 0.05 |
| D | 0.01 | 0.05 |
| B | 0.31 | 0.32 |

The results of the skin irritation test indicated that formulations A-D did not produce even minimal skin erythema, whereas formulation E produced at least minimal erythema in a high percentage (about 20%) of the subjects tested. Formulations A through D preferably provided skin irritation scores lower than a currently available anionic commercial formulation.

D. Time-kill Test

For time-kill testing, 1 ml of a culture of S. aureus (ATCC 6538) containing at least 1,000,000 colony forming units (CFU)/ml was mixed with 99 mls of a test formulation A-E. After 30 seconds, 1 ml of the mixture was removed, immediately neutralized using letheen broth, plated onto tripticase soy agar, grown for 48 hours at 37° C. and counted. The results, shown in Table 6 below, are expressed as log reduction (log CFU in control plate−log CFU in treated plate), corrected for dilution. Compositions exhibiting a log reduction of 2 or greater met the requirements of the invention.

TABLE 6
Time Kill Test

| Test Formulation | Log Reduction |
|---|---|
| A | >6 |
| B | 3.3 |
| C | >6 |
| D | 3.1 |
| E | <2 |

The results of the time kill test showed that test formulations A, B, C and D resulted in a greater reduction in the number of CFU (greater than a 2 log reduction) than commercial formulation E (less than a 2 log reduction). These results demonstrated that antimicrobial compositions containing non-cationic surface active components and quaternary ammonium compounds in a ratio of 21.9:1 were surprisingly effective in reducing or inhibiting bacterial growth compared to the commercial formulation. In this example, compositions containing glycerol resulted in a higher log reduction in bacterial growth than those containing decaglycerol. Thus, the combination of the present invention of cationic surfactant, nonionic surfactant and polyol provided a formulation that had acceptable antimicrobial activity and was non-irritating to the skin.

### EXAMPLE 2

The experiment described in this example was performed to evaluate the microbiocidal properties of a composition containing the quaternary ammonium compound benzethonium chloride as the antimicrobial agent, wherein the ratio of non-cationic surface active agents/cationic surfactant was about 6:1. The composition was prepared with the ingredients shown in Table 7.

TABLE 7

| Components | Weight % |
|---|---|
| Cationic Surfactant | |
| Benzethonium Chloride (50% solution) | 2.0 |
| Non-cationic surface actives | |
| Nonionic surfactants | |
| Dodecyl dimethyl amine oxide (30% | 6.5 |
| solution) | |
| PEO 9 C11-alcohol | 2.0 |
| Polyol | |
| Hydrogenated starch hydrolysate | 2.0 |
| Methoxycellulose | 1.5 |
| Deionized Water | to 100% |
| Non-cationic surface actives/cationic ratio | 6:1 |

A modified Cade test and time-kill tests as described in Example 1 were performed using commercial formulation E as a control. The results are shown in Table 8.

TABLE 8

| Formulation | Cade Test | Time-Kill |
|---|---|---|
| Test composition | >2.7 log | >4 logs |
| Commercial E | 0.3 log | <1 log |

These results demonstrated that a mixture of non-cationic surface actives and benzethonium chloride (cationic surfactant) at a ratio of about 6:1 had a much higher log reduction in the number of bacterial colonies remaining after washing, and thus was more effective in reducing or inhibiting bacterial growth than the commercial formulation.

### EXAMPLE 3

It is an object of the invention to provide a cleansing composition that has acceptable antimicrobial activity and minimizes skin irritation. Various amounts of nonionic surfactant (shown in Table 9) known to be less irritating to the skin were tested in a formulation containing BzCl to determine the optimal amount that could be added to provide additional skin cleansing properties, to retain acceptable microbiocidal activity of the formulation, and be nonirritating to the skin. Barlox 12 and Plantaren 818 are compatible with, that is do not deactivate, 1 mmole benzethonium chloride in a wide range of concentrations (about 10-30 mmole). However, Barlox 12 and Plantaren 818, when used alone, can be irritating to the skin. The nonionic surfactants N1-9, N25-12, 10-1-0 and 3-0.8-L are compatible with BzCl in only a very narrow range of concentrations (approximately 1.5-3 mmole). These surfactants, however, are desirable to supplement cleansing compositions because they are less irritating to the skin than other nonionic surfactants.

This example compared 16 compositions containing the following base ingredients:

0.5 wt % (active concentration) benzethonium chloride;

6.5 wt % of Barlox 12 (30% solution);

1.0 wt % Plantaren 818;

5 wt % of polyol: glycerol (odd numbered solutions) or decaglycerol (even numbered solutions); and

2 wt % sorbitol (40% solution).

Four additional nonionic surfactants, N1-9, N25-12, 10-1-0 and 3-0.8-L were added in the amounts shown in Table 9. The compositions were prepared as follows: Methocel 40-200 was pre-swelled to make a 4.0% solution in deionized water. 37.5% (w/w) of the 4.0% Methocel solution and the nonionic surfactants were combined; deionized water was added to each of the mixtures to 100%. The mixtures were blended until uniform. The formulations had a base ratio of non-cationic surface active agents/cationic surfactant of 17.5:1. The formulations were compared to a control formulation which contained the same base ingredients, however did not contain BzCl, and contained 2.5 wt % of each of glycerol and decaglycerol (sample #17); and Commercial E hand soap formulation containing triclosan (sample #18). The total % weight of the four nonionic surfactants, N1-9, N25-12, 10-1-0 and 3-0.8-L surfactants in the compositions ranged from 2% (Samples 1-4) to 4% (Samples 5-12) and to 6% (Samples 13-17).

Each of the formulations was challenged by a 30 second time kill test. 1 ml of a culture of S. aureus (ATCC 6538) containing at least 1,000,000 colony forming units (CFU)/ml was mixed with 99 ml of a test formulation. After 30 seconds, 1 ml of the mixture was removed and immediately neutralized using letheen broth, plated onto tripticase soy agar (with appropriate dilutions) and grown for 48 hours at 37° C. and counted. The results are shown in Table 9 and are expressed as log reduction (log CFU in control plate−log CFU in treated plate) corrected for dilution.

The results in Table 9 indicate that the addition of 2% (ratio of non-cationic to cationic surface active agents of 24:1) of either N1-9, N25-12 provided the highest log reduction in bacterial growth compared to all other samples. However, the addition of 4% of combinations of the nonionic surfactants (ratio of non-cationic to cationic surface active agents of 28:1) successfully maintained microbiocidal activity above the levels obtained for the control and commercial formulations. Although the addition of nonionic surfactant may not be deleterious to the mildness of the formulations, the results shown in Table 9 demonstrated that the addition of a total of 6 wt % of combinations of N1-9, N25-12, 10-1-0 or 3-0.8-L did not effectively control S. aureus in the time-kill test, and were comparable to the result obtained using the commercial standard. These results demonstrated that a ratio of non-cationic to cationic surface active agents of up to about 28:1 provided a suitable antimicrobial cleansing formulation.

TABLE 9

| | | | | | Ratio | |
|---|---|---|---|---|---|---|
| | Nonionic surfactant | | | | non-cationic | Log |
| Sample # | N 1-9 | N 25-12 | 10-1-0 | 3-0.8-L | / cationic | Reduction |
| 1 | 2 | — | — | — | 24.1 | 4.0 |
| 2 | 2 | — | — | — | | 4.6 |
| 3 | — | 2 | — | — | | 5.4 |
| 4 | — | 2 | — | — | | 5.6 |
| 5 | 2 | — | 2 | — | 28.1 | 2.0 |
| 6 | 2 | — | 2 | — | | 3.4 |
| 7 | — | 2 | 2 | — | | 1.9 |
| 8 | — | 2 | 2 | — | | 3.7 |
| 9 | 2 | — | — | 2 | | 1.6 |
| 10 | 2 | — | — | 2 | | 1.6 |
| 11 | — | 2 | — | 2 | | 2.2 |
| 12 | — | 2 | — | 2 | | 2.2 |
| 13 | 2 | — | 2 | 2 | 32.1 | <2 |
| 14 | 2 | — | 2 | 2 | | <2 |
| 15 | — | 2 | 2 | 2 | | <2 |
| 16 | — | 2 | 2 | 2 | | <2 |
| 17 | — | 2 | 2 | 2 | | <2 |

| | | |
|---|---|---|
| 18 | Commercial E | <2 |

Amounts are presented as w/w %

### EXAMPLE 4

This example illustrates the effect of pH on the antimicrobial activity of twelve cleansing formulations. Three sets of compositions A through L with ingredients shown in Table 10 were prepared by blending the ingredients, adding water to 100% and stirring until uniform. The three sets of samples differed as follows:

Series 1: Compositions were at pH 6.5; no EDTA or NaOH.

Series 2: Compositions with 2% EDTA added; pH adjusted to 8.0 with 50% NaOH.

Series 3: Compositions with 2% EDTA; pH adjusted to 6.5 with 50% NaOH.

The compositions had a ratio of non-cationic surface actives/cationic surfactant of from about 15:1 to 19:1.

TABLE 10

| | Quat | | Polyol | | Nonionic surfactact | | |
|---|---|---|---|---|---|---|---|
| # | BzCl | BACl | Gly | Deca-g | P818 | N25-12 | 10-1-O |
| A | 0.5 | — | 5 | — | 1 | 1 | 0.5 |
| B | — | 0.5 | 5 | — | 1 | 1 | 0.5 |
| C | 0.5 | — | 5 | — | 2 | 1 | 0.5 |
| D | — | 0.5 | 5 | — | 2 | 1 | 0.5 |
| E | 0.5 | — | 5 | — | 3 | 1 | 0.5 |
| F | — | 0.5 | 5 | — | 3 | 1 | 0.5 |
| G | 0.5 | — | — | 5 | 1 | 1 | 0.5 |
| H | — | 0.5 | — | 5 | 1 | 1 | 0.5 |
| I | 0.5 | — | — | 5 | 2 | 1 | 0.5 |
| J | — | 0.5 | — | 5 | 2 | 1 | 0.5 |
| K | 0.5 | — | — | 5 | 3 | 1 | 0.5 |
| L | — | 0.5 | — | 5 | 3 | 1 | 0.5 |

Samples were tested in a 3 0 second time-kill test against E. coli (ATCC # 11229) using the procedure described in Example 1. The results are expressed as log reduction and shown in Table 11.

TABLE 11

| | Series 1 | Series 2 | Series 3 |
|---|---|---|---|
| Sample | pH 6.5 (no EDTA) | pH 8.0 (EDTA) | pH 6.5 (EDTA) |
| A | 3 | 1 | 2.5 |
| B | 4 | 4 | 4 |
| C | 1.5 | 2 | 1.5 |
| D | 4 | 4 | 4 |
| E | 1 | 3 | 1 |
| F | 4 | 4 | 4 |
| G | 1 | 4 | 1 |
| H | 2.5 | 4 | 4 |
| I | 1 | 3 | 1.5 |
| J | 1 | 4 | 4 |
| K | 0.5 | 3 | 1.5 |
| L | 1 | 4 | 4 |

Adjustment of the pH was facilitated with the addition of EDTA without the loss of biocidal activity. Although benzalkonium chloride generally showed better control of the test organism than benzethonium chloride, formulations providing 3-4 log kill of the test organism were possible when the pH was adjusted to 8.0. It was also observed that, as the amount of Plantaren 818 was increased, the flash foam of the formulation during hand washing was increased, a desirable result.

### EXAMPLE 5

This example illustrates the effectiveness of the antimicrobial cleansing compositions of the present invention against three different bacterial species. The compositions contained varying amounts benzethonium chloride, 5 wt % decaglycerol (polyol) and 2 wt % Plantaren 818 (nonionic surfactant), 2 wt % EDTA and varying amounts of additional nonionic surfactants, Neodol 1-7, Neodol 9 1-6, Neodol 25-7, Neodol 25-12, Neodol 1-5, Neodol 1-9, Neodol 9 1-2.5, TDA-6, Tergitol 15-S-7, Tergitol 15-S-12, Tergitol 15-S-20, and Polyaldo 10-1-0 as shown in Table 12. The pH was adjusted to 7.5 by the addition of 50% NaOH after the addition of 2% EDTA. Deionized water was added to 100%.

A 30 second time kill test was run against each of the following organisms: S. aureus (ATCC #6538); E. coli (ATCC # 11229) and S. marcescens (ATCC # 14756). The results are provided as log kill and are shown in Table 12. All the compositions demonstrated at least one log reduction in bacterial growth against at least one of the organisms tested. Neodol 1-7 demonstrated the highest log reduction in bacterial growth of all three bacterial species overall compared to the other nonionic surfactants added.

TABLE 12

| | | | | Ratio | | | |
|---|---|---|---|---|---|---|---|
| | | | | non- | Log Reduction | | |
| | | | | cationic/ | E. | S. | S. |
| # | BzCl | Methocel | Nonionic Surfactant | cationic | coli | aureus | marcescens |
| 1 | 0.5 | — | 1.0 Neodol 1-7 | 16:1 | 1.5 | 4.0 | <1 |
| 2 | 0.5 | — | 1.0 Neodol 91-6 | 16:1 | <1 | 3.0 | 1.0 |
| 3 | 0.5 | — | 1.0 Neodol 25-7 | 16:1 | 1.0 | 2.0 | 1.5 |
| 4 | 0.5 | — | 1.0 Neodol 25-12 | 16:1 | <1 | 1.5 | 1.5 |
| 5 | 0.5 | — | 1.0 Neodol 1-5 | 16:1 | 1.0 | 2.0 | 0.5 |
| 6 | 0.5 | — | 1.0 Neodol 1-9 | 16:1 | <1 | 1.0 | 1.5 |
| 7 | 0.5 | — | 1.0 TDA-6 | 16:1 | 0.5 | 2.0 | 1.0 |
| 8 | 0.5 | — | 1.0 Tergitol 15-S-7 | 16:1 | 0.5 | 2.0 | 1.5 |
| 9 | 0.5 | — | 1.0 Tergitol 15-S-12 | 16:1 | 0.5 | 1.5 | 2.0 |
| 10 | 0.5 | — | 1.0 Tergitol 15-S-20 | 16:1 | 0.5 | 3.5 | 2.0 |
| 11 | 0.5 | — | 1.0 Neodol 91-2.5 | 16:1 | 1.5 | 4.0 | 2.0 |
| 12 | 0.75 | — | 1.0 Neodol 1-7 | 10.7:1 | 2.0 | 4.0 | 2.0 |
| 13 | 1.0 | — | 1.0 Neodol 1-7 | 8:1 | 5.0 | 4.0 | 2.0 |
| 14 | 1.5 | — | 1.0 Neodol 1-7 | 5.3:1 | 5.0 | 4.0 | 3.0 |
| 15* | 0.5 | — | 0 | 16:1 | 5.0 | 4.0 | 2.0 |
| 16 | 0.5 | 1.5 J12MS | 1.0 Neodol 1-7 | 16:1 | 2.5 | 2.5 | 2.5 |
| 17 | 0.5 | 1.5 40-100 | 1.0 Neodol 1-7 | 16:1 | 2.5 | 3.5 | 2.5 |
| 18 | 0.5 | 1.5 40-101 | 1.0 Neodol 1-7 | 16:1 | 2.5 | 3.5 | 1.5 |
| 19 | 0.5 | 1.5 40-202 | 1.0 Neodol 1-7 | 16:1 | 3.0 | 3.5 | 2.5 |
| 20 | 0.5 | 1.5 40-101 | 1.0 Neodol 1-7 and | 17:1 | 3.0 | 4.0 | 3.0 |
| | | | 0.5 Polyaldo 10-1-O | | | | |
| 21 | — | 1.5 40-101 | 1.0 Neodol 1-7 | — | 0 | 0 | 0 |

*contained 3% P818

What is claimed is:

1. An aqueous rinse off antimicrobial cleansing composition, comprising from about 3 to about 25 wt % of non-aqueous constituents, said non-aqueous constituents comprising from about 0.2 to about 4 wt % of a quaternary ammonium cationic surfactant; from about 2 to 10 wt % of a nonionic surfactant; and from 1 to 10 wt % of a polyol, wherein the weight ratio of nonionic surfactant and polyol to cationic surfactant is from at least 4:1 to about 28:1 wt %.

2. The composition of claim 1, wherein the weight ratio of nonionic surfactant and polyol to cationic surfactant is from at least 4:1 to about 24:1.

3. The composition of claim 1, wherein the quaternary ammonium cationic surfactant is a dialkyl dimethyl ammonium compound, an alkylbenzylammonium compound, an alkyltrimethyl ammonium compound, a benzalkonium compound or a benzethonium compound.

4. The composition of claim 3, wherein the quaternary ammonium cationic surfactant is benzalkonium chloride or benzethonium chloride.

5. The composition of claim 1, wherein the polyol is glycerol, polyglycerol, sorbitol or hydrogenated starch hydrolysate.

6. The composition of claim 5, wherein the polyol is glycerol or decaglycerol.

7. The composition of claim 1, wherein the nonionic surfactant is an alkyl polyglucoside, an alkyl phenol ethoxylate, an alcohol ethoxylate, an amine oxide, a polyglycerol ester or mixtures thereof.

8. The composition of claim 7, wherein the nonionic surfactant is an ethoxylate of a linear alcohol having twelve or less carbon atoms or an amine oxide having an alkyl group having from 8 to 14 carbon atoms.

9. A method for providing residual microbiocidal effectiveness on human skin, which comprises applying the composition of claim 1 on the skin in a microbiocidal effective amount.

10. An aqueous composition comprising about 0.2 to 4 parts of a quaternary ammonium cationic surfactant; about 2 to 10 parts of a nonionic surfactant; and about 1 to 10 parts of a polyol, wherein the weight ratio of nonionic surface active agents and polyol to cationic surfactant is at least 4:1 to about 28:1.
